(19) United States
(12) Patent Application Publication
(10) Pub. No.: US 2006/0217787 A1
(43) Pub. Date: Sep. 28, 2006

(54) Light therapy device

(75) Inventors: Donald E. Olson, Rochester, NY (US); Andrew F. Kurtz, Macedon, NY (US); Robert P. Bourdelais, Pittsford, NY (US); Cheryl J. Brickey, Webster, NY (US)

Correspondence Address: Mark G. Bocchetti;Patent Legal Staff, Eastman Kodak Company, 343 State Street, Rochester, NY, 14650-2201, US

(73) Assignee: Eastman Kodak Company

(21) Appl. No.: 11/087,300

(22) Filed: Mar. 23, 2005

(51) Int. Cl. A61N 5/06

(52) U.S. Cl. 607/88; 607/89

(57) ABSTRACT

A light therapy device (40) for delivering light energy to a portion of a patient's body comprises a light source (115). The light source comprises one or more light emitters (122) for providing input light. A light coupling means (80) directs the input light into a light guide (140). A flexible optically transparent light guide material comprises the light guide. A light extraction means (75) is applied to a surface of the light guide material. The light extraction means is positioned to provide light therapy treatment to one or more localized areas of the patient's body. A control means controls a light dosage relative to intensity, wavelength, modulation frequency, repetition, and timing of treatments.

### FIELD OF THE INVENTION

The invention relates generally to a light therapy device and in particular, to a light therapy device for use in close proximity, or in contact with, the skin or a patient.

### BACKGROUND OF THE INVENTION

The term “phototherapy” relates to the therapeutic use of light, and the term “illuminator” or “light therapy device” or “phototherapy device” refers to a device that is generally intended to be used externally to administer light to the skin of a patient for therapeutic purposes.

External light therapy has been shown to be effective in treating various medical conditions, for example, seasonal affective disorder, psoriasis, acne, and hyperbilirubinemia common in newborn infants. Light therapy has also been employed for the treatment of wounds, burns, and other skin surface (or near skin surface) ailments. As one well-known example, light therapy can be used to modify biological rhythms in humans, such as circadian (daily) cycles that affect a variety of physiologic, cognitive, and behavioral functions. Light therapy has also been used for other biological treatments that are less recognized. For example, in the late 1800's, Dr. Niels Finsen found that exposure to ultraviolet radiation aggravated smallpox lesions. Thus, he illuminated his patients with light with the UV filtered out. Dr. Finsen further discovered that exposure with the residual red light sped healing in recovering smallpox victims. Finsen also determined that ultraviolet radiation could be used to heal tuberculosis lesions. As a result, in 1903, Dr. Finsen was awarded a Nobel Prize for his use of red light therapy to successfully treat smallpox and tuberculosis.

In the 1960's and 1970's researchers in Eastern Europe undertook the initial studies that launched modern light therapy. One such pioneer was Endre Mester (Semmelweiss Hospital, Budapest, Hungary), who in 1966, published the first scientific report on the stimulatory effects of non-thermal ruby laser light (694 nm) exposure on the skin of rats. Professor Mester found that a specific range of exposure conditions stimulated cell growth and wound healing, while lesser doses were ineffective and larger doses were inhibitory. In the late 1960's, Professor Mester reported the use of laser light to treat non-healing wounds and ulcers in diabetic patients. Mester's 70% success rate in treating these wounds lead to the development of the science of what he called “laser biostimulation.”

Photodynamic therapy (PDT) is one specific well-known example of light therapy, in which cancerous conditions are treated by a combination of a chemical photo-sensitizer and light. Typically in this instance, several days before the light treatment, a patient is given the chemical sensitizer, which generally accumulates in the cancerous cells. Once the sensitizer concentrations in the adjacent non-cancerous cells falls below certain threshold levels, the tumor can be treated by light exposure to destroy the cancer while leaving the non-cancerous cells intact.

As compared to PDT, light therapy, as exemplified by Professor Mester's pioneering work, involves a therapeutic light treatment that provides a direct benefit without the use of enabling external photo-chemicals. Presently, there are over 30 companies world wide that are offering light therapy devices for a variety of treatment applications. These devices vary considerably, with a range of wavelengths, power levels, modulation frequencies, and design features being available. In many instances, the exposure device is a handheld probe, comprising multitude light emitters; that can be directed at the patient during treatment. The light emitters, which typically are laser diodes, light emitting diodes (LEDs), or combinations thereof, usually provide light in the red-IR (˜600-1200 nm) spectrum, because the tissue penetration is best at those wavelengths. In general, both laser light and incoherent (LED) light seem to provide therapeutic benefit, although some have suggested that lasers may be more efficacious. Light therapy is recognized by a variety of terms, including low-level-laser therapy (LLLT), low-energy-photon therapy (LEPT), and low-intensity-light therapy (LILT). Despite the emphasis on “low” in the naming, in actuality, many of the products marketed today output relatively high power levels, of up to 1-2 optical watts. Companies that presently offer light therapy devices include Thor Laser (United Kingdom), Omega Laser Systems (United Kingdom), MedX Health (Canada), Quantum Devices (United States) and Lumen Photon Therapy (United States).

Many different examples of light therapy and PDT devices are known in the patent art. Early examples include U.S. Pat. No. 4,316,467 (Muckerheide) and U.S. Pat. No. 4,672,969 (Dew). The most common device design, which comprises a hand held probe, comprising at least one light emitter, but typically dozens (or even 100) emitters, that is attached to a separate drive controller, is described in numerous patents, including U.S. Pat. No. 4,930,504 (Diamantapolous et al.); U.S. Pat. No. 5,259,380 (Mendes et al.); U.S. Pat. No. 5,464,436 (Smith); U.S. Pat. No. 5,634,711 (Kennedy et al.); U.S. Pat. No. 5,660,461 (Ignatius et al.); U.S. Pat. No. 5,766,233 (Thiberg); and U.S. Pat. No. 6,238,424 (Thiberg).

One shortcoming of the probe type laser therapy device is that it requires the clinician, or perhaps the patient, to actively apply the laser light to the tissue. Typically, the clinician holds the light therapy probe, aims the light at the tissue, and operates the device according to a treatment protocol. As a result, the laser therapy devices are often designed to emit high light levels, in order to reduce the time a clinician spends treating an individual patient to a few minutes or less, whether the application conditions are optimal or not. Additionally, in many such cases, the patient is required to travel to the clinician's facility to receive the treatment. Because of this inconvenience, patients are typically treated only 1-3 times/week, even if more frequent treatments would be more efficacious.

Certainly, these shortcomings with the handheld probes have been previously identified. For example, Laser Force Therapy (Elizabeth, Colo.) offers a disk-shaped probe (the “Super Nova”) that can be strapped onto the patient. While this is a potential improvement, the device does not conform to the shape of the tissue being treated. As an alternate approach, a variety of self-emissive light bandages have been suggested, in which a conformal pad having a light emitting inner surface is strapped directly on the patient. Since the patient can wear the device, perhaps under their clothes for a prolonged period of time, the convenience limitations of the handheld probe may be overcome.

As a first example, U.S. Pat. No. 6,569,189 (Augustine et al.) provides a heat therapy bandage that uses IR blackbody radiation generated from electrical resistance in circuit trace within the bandage. In this case, since the emitted light is broadband IR (nominally 3-30 microns), this bandage does not enable the use of specific illumination optical wavelengths that have been suggested to be optimal for treating various conditions. In particular, the wavelengths provided by this device may not advantageously activate the known photo-acceptor molecules in cells. Moreover, this device does not offer a means to vary the light spectrum in any useful way.

As an example, Omnilight (Albuquerque, N. Mex.) offers the Versalight pads, which combine a controller (such as the VL3000) with a pad, where the pads comprise a multitude of discrete LEDs imbedded in a neoprene-covered foam. Bioscan Inc. (Albuquerque, N. Mex.) offers a similar suite of products for veterinary applications. In both cases, the products typically comprise a mix of IR and red LED emitters, arranged in a pattern across the pad. These devices are described in U.S. Pat. No. 4,646,743 (Parris), which teaches conformal pad light therapy devices in which an array of diodes is imbedded in pliable foam. Several other similar devices are known in the prior art, including:
U.S. Pat. No. 5,358,503 (Bertwell et al.), which provides a conformal pad utilizing tightly packed LEDs, which is placed in contact with the tissue, so as to provide both light and thermal treatments.
U.S. Pat. Nos. 5,616,140 and 5,989,245 (both to Prescott), which provides a conformal bandage comprising laser diodes and flexible circuitry fabricated within a multi-layer pad.
U.S. Pat. No. 5,913,883 (Alexander et al.), which provides a conformal therapeutic facial mask comprising a plurality of LEDs held off of the tissue by spacer pads.
Other prior art patents that provide for conformal light therapy pads with discrete light emitters mounted to a pad substrate include U.S. Pat. No. 6,187,029 (Shapiro et al.); U.S. Pat. No. 6,290,713 (Russell); U.S. Pat. No. 6,443,978 (Zharov); and U.S. Pat. No. 6,743,249 (Alden).

While these various patents provide designs for conformal light therapy pads, these devices are disadvantaged by their awkward construction, which typically involves mounting some number of rigid discrete diodes (lasers or LEDs) within a conformal pad, accompanied by the required drive circuitry and thermal management means. As a result, these devices are encumbered by some manufacturing difficulties that affect unit cost, and likely limit the potential that these devices could become ubiquitous, if not disposable.

As an alternate approach, there are a variety of technologies being developed for self emissive devices, such as organic light emitting diodes (OLEDs), polymer light emitting diodes (P-LEDs), and thin film flexible electroluminescent sources (TFELs), which could readily enable volume production. As an example, U.S. Pat. No. 6,096,066 (Chen et al.) teaches a flexible LED array on a thin polymer substrate, with addressable control circuitry, slits for perspiration, and the use of LEDs, which could be replaced with OLEDs. Similarly, U.S. Patent Application Publication No. 2004/0111132 (Shenderova) discloses a thin film electroluminescent (TFEL) phototherapy device based on high field electroluminescence (HFEL) or OLED technologies. Certainly, light therapy bandages based on these technologies have several potential advantages, including volume production and customizable temporal and spatial control from the addressing circuitry. However, even in the target display markets (laptop computers, television, etc.) OLED technologies are not yet sufficiently mature to support volume production. Also, while self emissive light bandages will not be encumbered by lifetime issues and the resolution requirements imposed on the display market, such bandage type devices will have their own issues (minimizing toxicity, providing sufficient output power or IR output light) that will likely effect the appearance of such devices in health markets.

Therapeutic light pads have also been developed using woven bundles of optical fibers. Such devices are typically marketed for use in treating jaundice in infants. One example is the Biliblanket Plus, offered by Ohmeda Medical (Baltimore, Md.), which uses a high intensity halogen lamp, mounted in a controller and light coupled into a fiber bundle. The fiber bundle, nominally comprising 2400 individual optical fibers, is configured into a woven pad, in which the bends in the optical fibers cause local breakdown in total internal reflection, so that light is coupled out of the fiber over the full surface area of the pad. Another company, Respironics (Murrysville, Pa.), offers a similar system, the Wallaby Phototherapy System, for neonatal care of jaundice. The basic concept for a woven fiber-optic illuminator is described in U.S. Pat. No. 4,234,907 (Daniel).

This type of medical light therapy pad, using an illuminator comprising a woven mat of optical fibers, is described in prior art patents U.S. Pat. No. 5,339,223 (Kremenchugsky et al.) and U.S. Pat. No. 5,400,425 (Nicholas et al.), both assigned to Ohmeda Inc. For example, the prior art light therapy device of U.S. Pat. No. 5,400,425, shown in FIG. 1, comprises a woven fiber-optic pad 10 connected by a fiber-optic cable 12 to a drive unit 14 that houses a source of light. The fiber-optic cable 12 has a protective coating of a plastic material such as vinyl and contains a plurality of individual optical fibers, not shown in FIG. 1, which transmit the light from the drive unit 14 to the woven fiber-optic pad 10 for emission toward the infant. A connector 16, affixed to an end of fiber-optic cable 12, positions the cable to receive light energy from a light source (internal to the drive unit 14 and not shown). The light source is typically a quartz halogen lamp, although xenon lamps, tungsten halogen lamps, LEDs, and other light sources can be used. Also within the drive unit 14 are the various electrical components and optical components, the latter including optical filters to obtain the desired wavelength of the light radiation delivered to the fiber-optic cable 12 in the range of about 400 to 550 nanometers. Other filters may filter out infrared and UV radiation spectrums from the light radiation delivered. The drive unit 14 is also shown equipped with a controller 20 and a display 22 mounted on the front panel 24, which may facilitate intensity and frequency modulation of the light. In combination with the drive unit 14, fiber-optic pad 10 comprises a plurality of optical fibers woven so as to emit light energy for phototherapy. U.S. Pat. No. 6,494,899 (Griffin et al.), assigned to Respironics Inc., provides an improved device in which the lamp source can be automatically changed after a lamp failure.

U.S. Pat. No. 4,907,132 (Parker) provides an improved woven fiber-optic light therapy device where the pad is designed for improved light efficiency and controlled output. Accordingly, the uniformity of illumination of a pad may be varied by varying the shape of the optical fiber disruptions or bends and/or the spacing between such disruptions or bends as by varying the pattern and tightness of the weave or by varying the proportion of optical fibers to other material in the weave. U.S. Pat. No. 4,907,132 also provides that the fiber-optic pad may have a transparent coating laminated applied to the outer surfaces of the disruptions or bends on one or both sides of each optical fiber layer. The coating is intended to cause changes in the attenuation of light being emitted from the pad. The coating increases the overall optical efficiency of the pad by causing attenuation changes only where the light normally escapes from the disruptions or bends of the woven optical fiber panel. While control of the pattern and tightness weave certainly will effect light emission over the pad, such customization likely occurs at the factory, rather than at a clinic or even in the home. The other approach, with the transparent overcoat layers, may lend itself to customization at the treatment facility. However, while the over coat seems to offer effective control of the light output, fiber-optic light emission at the bends is largely controlled by the radius of the bends and the core and cladding refractive indices, and applying a transparent coating onto the cladding may only have a secondary effect on the light emission characteristics.

While such systems may have achieved certain degrees of success in their particular applications, there is yet a need for a low cost flexible light therapy device that can be safely, readily, and comfortably used. In particular, it would be desirable if a clinician could modify the output of a light therapy bandage device to provide light at a desired treatment area, but not elsewhere. As an example, a pressure ulcer is typically a localized wound that affects several square inches of tissue. An overlaying bandage, whether a light therapy device, an alginate based bandage, a hydrocolloid dressing, or some other type of dressing, tend to extend over large areas of the surrounding tissue. While a clinician may want some illumination of the surrounding area, that desired illumination area may still be much smaller than the overall bandage size. Moreover, it would be desirable to allow a clinician to customize the light therapy treatment area to follow the typically irregular shape of the treatment area (a pressure ulcer, a burn, etc.). In particular, it would be desirable if the clinician can customize the light emission area to match the wound area during a given visit to the patient, rather than having to make a return trip. Additionally, it would be desirable if the customized light therapy treatment device can be readily changed upon subsequent visits to the patient. Finally, it would be desirable if such a light therapy device could be fabricated using high volume manufacturing technologies that are already in place today.

### SUMMARY OF THE INVENTION

Briefly, according to one aspect of the present invention a light therapy device for delivering light energy to a portion of a patient's body comprises a light source. The light source comprises one or more light emitters for providing input light. A light coupling means directs the input light into a light guide. A flexible optically transparent light guide material comprises the light guide. A light extraction means is applied to a surface of the light guide material. The light extraction means is positioned to provide light therapy treatment to one or more localized areas of the patient's body. A control means controls a light dosage relative to intensity, wavelength, modulation frequency, repetition, and timing of treatments.

These objects are given only by way of illustrative example, and such objects may be exemplary of one or more embodiments of the invention. Other desirable objectives and advantages inherently achieved by the disclosed invention may occur or become apparent to those skilled in the art. The invention is defined by the appended claims.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 shows a perspective view of a prior art light therapy device comprising a fiber-optic mat type illuminator and a drive unit.

FIG. 2 shows a diagrammatic view of a light therapy device in accordance with the present invention.

FIGS. 3a and 3b show side views of the light therapy device of FIG. 2 showing application of a light extraction layer.

FIGS. 4a, 4b, 4c and 4d show top views of the light guide substrate of the light therapy device of the present invention, with different configurations of light extraction layers.

FIG. 5a shows a top view of an alternate embodiment of a portion of the light therapy device of the present invention.

FIG. 5b shows a side view of an alternate embodiment of a portion of the light therapy device of the present invention.

FIG. 5c shows a perspective view of an alternate embodiment of a portion of the light therapy device of the present invention.

FIG. 6a shows a top view of an alternate embodiment of the light therapy device of FIG. 2 of the present invention.

FIG. 6b shows a side view of the FIG. 6a alternate embodiment of the light therapy device of the present invention.

FIG. 7 shows a perspective view of a light therapy device of the present invention in position to apply treatment to a limb of a patient.

FIGS. 8a and 8b show views of alternate configurations for portions of the light therapy device of the present invention.

FIG. 9 shows a perspective view of a light therapy device of the present invention in position to apply treatment to the face of a patient.

### DETAILED DESCRIPTION OF THE INVENTION

The following is a detailed description of the preferred embodiments of the invention, reference being made to the drawings in which the same reference numerals identify the same elements of structure in each of the several figures.

The present invention provides a flexible light therapy device having a plurality of applications, including but not limited to, the treatment of seasonal affective disorder, psoriasis, acne, diabetic skin ulcers, pressure ulcers, and hyperbilirubinemia common in newborn infants. The present invention delivers light energy by means of a flexible member that can be placed in contact with the skin of a patient. The present invention comprises a light guide bandage, in which light is input coupled into the light guide, trapped within by reflection, and emitted in accordance with a light extraction layer. The light extraction layer can be custom applied, such that the light is emitted nominally onto the area requiring treatment but not elsewhere over the output face of the bandage. The device is nominally designed to be readily worn by the patient for a prolonged time period, and is potentially disposable thereafter.

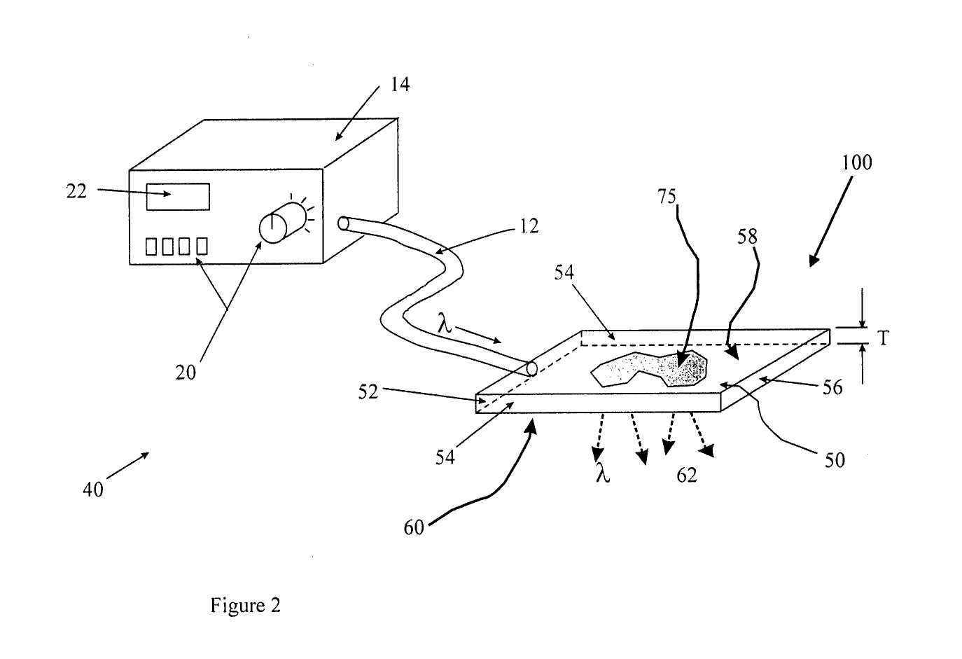

FIG. 2 generally illustrates a diagrammatic view of a first embodiment of a light therapy device 40 in accordance with the present invention. As illustrated in FIG. 2, light therapy device 40 comprises a drive unit 14 with an internal light source (not shown), a fiber-optic cable 12 to couple light from the light source into the light therapy pad. The drive unit 14 can be equipped with a display 22 and a controller 20, which facilitates setting of treatment parameters such as light intensity, frequency, wavelength, modulation, and repeat treatment timing. While fiber-optic cable 12 is nominally identified as containing a fiber-optic bundle, other flexible light piping means can be used, such as liquid light pipe or solid dielectric light pipe.

The light guide therapy pad 100 depicted in FIG. 2 is nominally a transparent optical sheet, wherein light is input coupled at input surface 52. Light is then trapped within the light guide substrate by reflections off of outer surface 58, inner surface 60, side surfaces 54, and end surface 56. A portion of the light trapped within the light guide substrate 50 encounters light extraction layer 75, which re-directs the light, so that it is coupled out of the light guide substrate 50 as therapeutic light 62. Light guide substrate 50 is nominally a non-woven sheet material, such as a flexible transparent elastomeric polymer polyurethane. Other polymers could be used, such as acetate sheets, although the substrate could also be made with a thin optical glass. The light is nominally trapped within the light guide substrate 50 by total internal reflection off of outer surface 58, inner surface 60, and side surfaces 54. End surface 56 will likely utilize a reflective layer as mirror layer 82. This reflective layer could be a dielectric (MgFl, for example) or a metal (aluminum, for example) layer to reflect the incident light back into the light guide substrate 50. More likely a commercial reflectance film, such as 3M Vikuiti enhanced specular reflection film (ESR) would be used, with an intermediate adhesive layer used to attach the film to light guide substrate 50. Light guide therapy pad 100 is nominally a wave-guide, with a thickness T that supports multi-mode light guiding along its length and across its width. Light extraction layer 75, can for example, be an optical diffuser such as a sheet white matte reflective beads which can be cut to a shape corresponding to the treatment area and then externally applied to the light guide substrate 50.

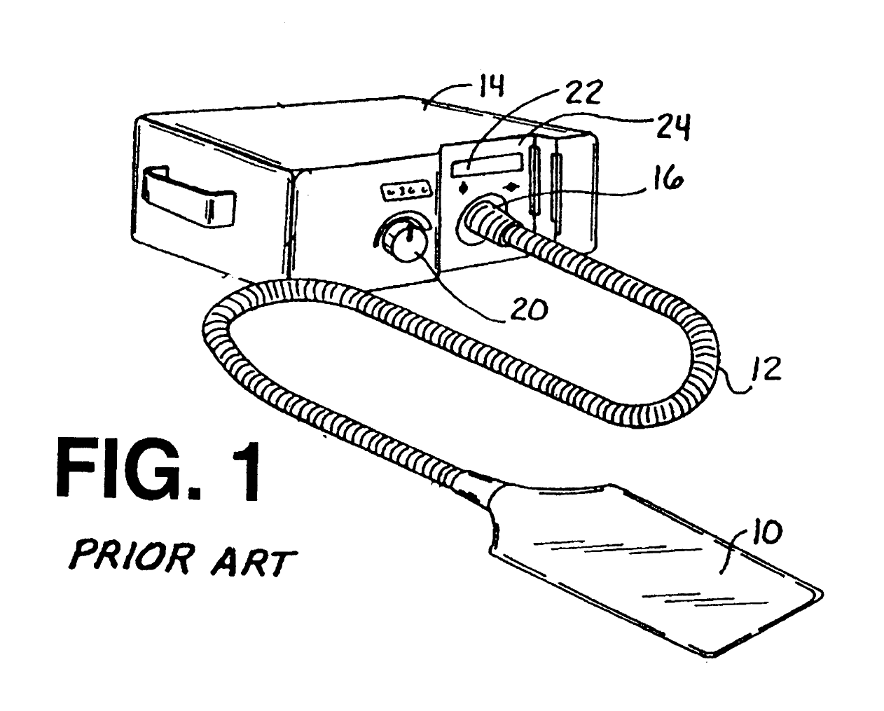

Although to the casual observer, light guide therapy pad 100 of FIG. 2 may appear similar to the prior art fiber-optic pad 10 discussed with respect to FIG. 1, there are several important differences. Light guide therapy pad (or bandage) 100 is not constructed from a multitude of woven optical fibers, but instead comprises a substrate that is a nominally homogeneous optical sheet material. The distribution of the therapeutic light emerging from the prior art fiber-optic pad 10 largely relies on the optical coupling into the individual optical fibers, the distribution of those optical fibers, and the manner and frequency in which bends are imparted to the optical fibers when they are woven. In the case of the light guide therapy pad 100 of the present invention, the distribution of the therapeutic light 62 emerging from the light guide substrate 50 largely depends on the light coupling at the input surface 52, the use of any beam shaping optics at the input surface, and the properties and dimensions of the light extraction layer 75. These differences will become more apparent during the subsequent descriptions of the present invention.

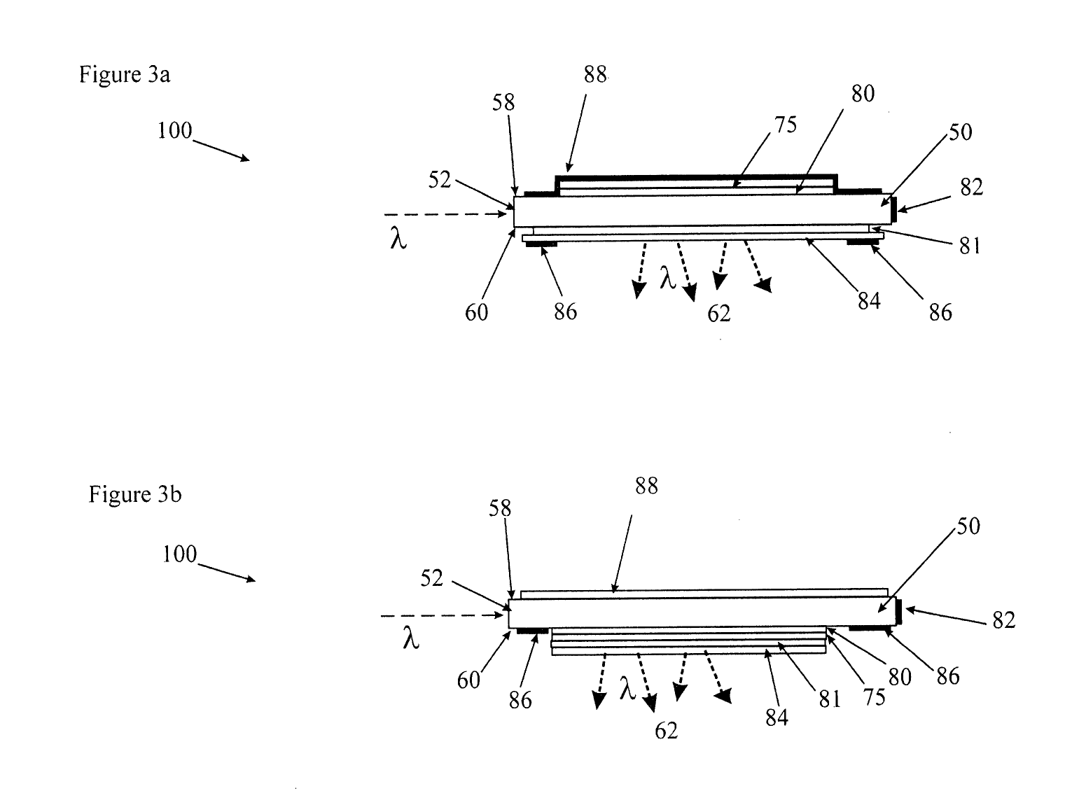

FIGS. 3a and 3b depict cross sectional views of two basic constructions for the light guide therapy pad 100. In the case of FIG. 3a, light guide therapy pad 100 comprises light guide substrate 50, with a light extraction layer 75 mounted on outer surface 58. Light extraction layer 75 is nominally a reflective optical diffuser, such as the white reflective diffusers commonly used in the manufacture of laptop computer displays. An exemplary optical diffuser that might be used for light extraction layer 75 is the LTO series reflective diffuser from Tsujiden Co. Ltd. (Japan). In this case, light that incident into the light guide substrate 50 reflects internally until it encounters the diffusing light extraction layer 75. Most of this incident light then diffusely reflects from the light extraction layer 75, back towards the light guide substrate 50, through the thickness T of the light guide substrate 50, and exits out the inner surface 60 as therapeutic light 62. Of course, some of the diffusely reflected light will be reflected such that it remains trapped within the light guide substrate 50. After multiple reflections, a portion of that light will again encounter the light extraction layer 75, where it will again be diffusely reflected, and may yet contribute to the therapeutic light 62 emerging from the device. FIG. 3a also depicts light guide therapy pad 100 as constructed with an optional optical coupling layer 80 between light extraction layer 75 and substrate 50. This layer could have both refractive index matching properties and adhesive properties to enhance the efficiency and uniformity of the optical diffusion.

Other properties of a light guide therapy pad 100 are depicted in FIG. 3a. For example, end surface 56 can be coated with a mirror layer 82 to prevent the light from spilling out the ends of the light guide substrate 50. Side surfaces 54 could also be provided with a mirror layer (such as 3M ESR film) rather than relying on total internal reflection to provide the light trapping. Surfaces 54, 58, and 60 could also be coated with scattered matte beads to provide miniature standoffs, so that other applied layers and materials could be kept from defeating the total internal reflection of the light guide outside the treatment area. Input surface 52 could likewise have a mirror coating, aside from any clear apertures that are provided for the input light to enter the light guide substrate 50. A cover 88 can be provided on the outer surface 58. Cover 88 could be a coating or a sleeve, made of gauze or some other material. It could serve several functions, including to protect the light guide therapy pad 100 from damage and contamination (from pathogens; and optically (to prevent degradation of the reflecting and diffusing properties of the bandage surfaces), or to fasten the therapy bandage to other bandage elements (such as straps), etc. Cover 88 could have multiple properties and functions; for example on the outer edges of outer surface 58, it could comprise one or more Velcro strips for attaching light guide bandage 100 to other bandage elements. Cover 88 could also have localized properties elsewhere relative to outer surface 58, such as for protection of the optical properties of the light guide, etc. Cover 88 could also provide a non-stick surface, so the light guide therapy pad 100 does not catch on clothing the patient may wear over the pad. Prior art patents, such as U.S. Pat. No. 5,759,570 (Arnold) and U.S. Pat. No. 6,528,697 (Knutson et al.) suggest approaches for constructing composite modular bandages that might be appropriate for the light therapy bandage of the present invention.

Light guide substrate 50 may also have layers and coatings on the inner surface 60. For example, a tissue interface layer 84 can be provided, which could have antibiotic properties or bio-sensing capabilities. For example, tissue interface layer 84 could have topical agents that fight infection (including anti-biotic silver), encourage epithelialization or other tissue healing activities, or amplify the effects of light therapy. In the case of bio-sensing, the bio-sensor features might detect a bio-physical or bio-chemical condition of the treatment area, which can then be used as input to guide further treatments. For example, the biosensors might detect the presence or absence of certain pathogens or enzymes associated with infections, or other enzymes and proteins associated with healing. Light guide bandage 100 could also be equipped with a sensing means that changes color relative to time to indicate the time (or amount of exposure) and thereby indicates an end to a given therapy session. For example, biosensors could be used to look for bio-chemical indications of the effective dosage applied. Alternately, optical sensors could detect the backscattered light as measure of the optical dosage delivered. The end of session control could then be manual or automatic.

Light guide substrate 50 may also have adhesive layers 86 on the inner surface 60, which might help to attach the light guide therapy pad 100 directly onto the tissue, or to other bandage elements. Alternately, adhesive layers 86 could represent other types of attachment means, such as Velcro, which could be used to fasten the light guide therapy pad 100 to other bandage elements. Other cover layers (not shown) could also be provided, to aid in assembly of a composite bandage, incorporating other bandage technologies, such as hydrocolloidal or alginate type dressings, silver based anti-biotic dressings, etc. Obviously, the addition of such dressings should minimally interfere with the use of the light therapy bandage. Also, contact with the patient's body can require disposing device 40 within a hygienic enclosure/sheath/sleeve. That is, it is recognized that there may be applications (e.g., instances of potential infections) wherein it may be desired to reduce the potential spread of germs. As such, it may be desirable to employ a hygienic sleeve, as known to those skilled in the art (e.g., as used with digital thermometers), for example, a transparent material such as a polymer sheet or bag. The sleeve might then be comprised of an anti-bacterial material. Alternatively, light guide bandage 100 might include an anti-bacterial layer disposed on the surface intended for contact with the patient's skin. The adhesive layer 86 could also be spongy, to provide better comfort for the patient when the light therapy bandage is worn.

An alternate cross-sectional construction of light guide therapy pad 100 is shown in FIG. 3b. In this case, a transmissive light extraction layer 75 is provided on the inner surface 60 of light guide substrate 50. Light extraction layer 75 could have a micro-structured optical surface, with micro-prisms, micro-lenses, or other features, which will cause incident light (from internal to light guide substrate 50) to refract, diffract, and/or scatter out of the light guide substrate 50 and emerge as therapeutic light 62. An exemplary light extraction layer could be a brightness enhancement film (BEF) from 3M. Potentially, extraction layer 75 could be formed with micro-structured light extraction features that are directly embossed or patterned into either outer surface 58 or inner surface 60 of the light guide substrate 50. Other complementary layers, such as cover 88, mirror layer 82, adhesive layers 86, and tissue interface layer 84 are shown for completeness.

Ongoing research into light therapy has also suggested that it can be advantageous to illuminate the tissue being treated with polarized light, as compared to non-polarized light. Therefore it may be beneficial to equip the light guide therapy device 100 of the present invention with the ability to polarize light. To exemplify this, the conceptual devices of FIGS. 3a and 3b are shown to possibly comprise other optical layers 81. For example, other optical layers 81 could comprise a polarizing film or a polarization conversion film, as are used in the display industry to build laptop and television displays. Other optical layers 81 could have other desirable properties; and for example be optical filters or be photo-chemically active, and react (change color) in response to bio-chemically emitted light emerging from the tissue in connection to some ongoing biological process. As such, other optical layers 81 could serve as a diagnostic device, similar to the previously mentioned biosensors. The clinician could be equipped with light guide substrates lacking such other optical layers 81, and apply them as needed, or the dielectric substrates could be pre-fabricated with such layers, and the clinician could be offered a range of light guides with different properties, and choose accordingly.

It should be understood that the cross-sectional views of FIGS. 3a and 3b are meant to be illustrative of the general concepts, and do not represent the actual relative physical size of the various constituent layers and components. Other figures are intended to be similarly illustrative.

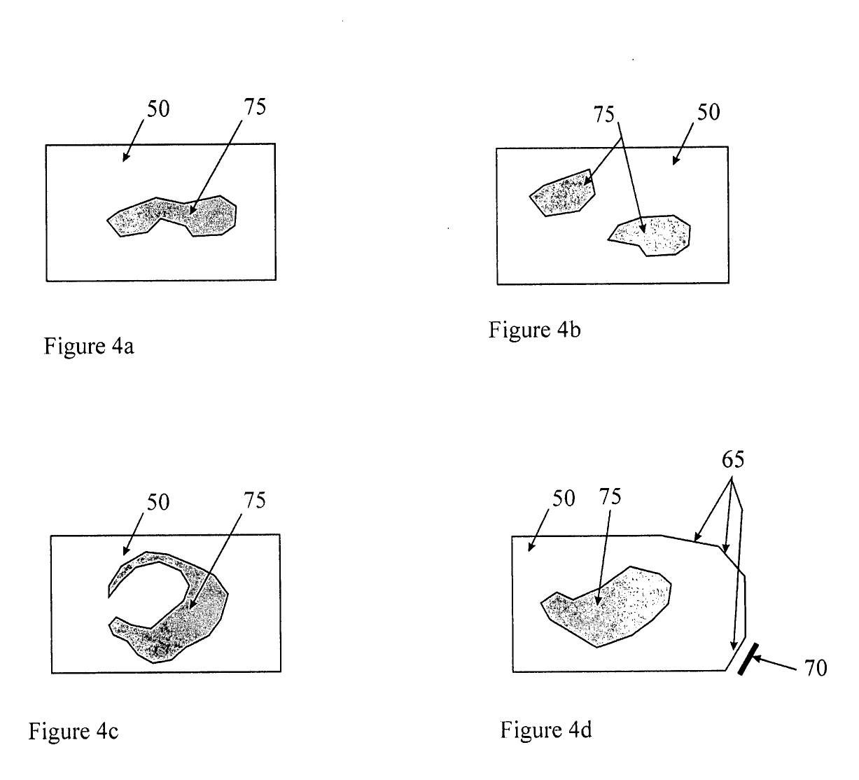

Another aspect of light guide therapy pad 100 is depicted in FIGS. 4a-4c. In particular, these figures illustrate the intention that light extraction layer 75 can be optimized to illuminate a given treatment area or areas, even if they are irregular in shape. In particular, FIG. 4b depicts a light guide therapy pad 100 with two light extraction layers 75 disposed on a surface. It is generally preferable to pattern light extraction layer 75 in the general shape of the treatment area, and to illuminate the wounded tissue or an area somewhat larger, rather than to illuminate the tissue over the entire surface area of the light guide therapy pad 100, as there will then be greater efficiency in delivering light to the wounded area. In some cases, depending on the size of the wound and the available bandages, or the type of wound, the light extraction layer 75 could cover nearly the entire surface of light guide therapy pad 100.

Considering FIGS. 4a-4c, a clinician might measure directly, or via a digital camera, the boundary regions of a wound. This data would then be transferred over to a sheet of light extraction layer 75 material, where the pattern of the boundary regions would be generally replicated. Depending on the construction of the light guide therapy pad 100, the location of the patient (in a clinic or hospital, or at home) and the capabilities of the clinician, the pattern transfer to the light extraction layer 75 might happen in situ with the patient, or remotely. For example, in the remote case the digital image data for a wound might be transferred to another location, where the light extraction layer 75 could be printed or cut from a material. The final assembly of the bandage could then occur at that location. If the patterning of the light extraction layer 75, and the attachment thereof to the light guide substrate 50, is a simple process, the customized light guide therapy pad 100 could be completed by the clinician in the field.

Although it may be optimal for a clinician to customize the light extraction layer 75 to the wound area, using a digital camera (for example), it may also be that the practicalities of image capture and transfer, and completion of the bandage 100 may prove too cumbersome for some circumstances. Thus, it should be understood that a clinician could be provided with a set of pre-shaped and pre-sized light extraction layers 75 (for example, with round and/or oval shapes). The clinician could then choose the light extraction layer 75 that most closely resembles the desired treatment area. The clinician could then apply the light extraction layer 75 to light guide substrate by the appropriate methods to complete the bandage preparation. Alternately, a range of bandages 100 could be pre-assembled at the factory, comprising a range of differently shaped and sized light extraction layers 75. The clinician could then select the most appropriate bandage 100 from the selection available. Assuming that the bandage cost is sufficiently low, then the burden of having a selection of pre-fabricated bandages available to one or more clinicians could be manageable.

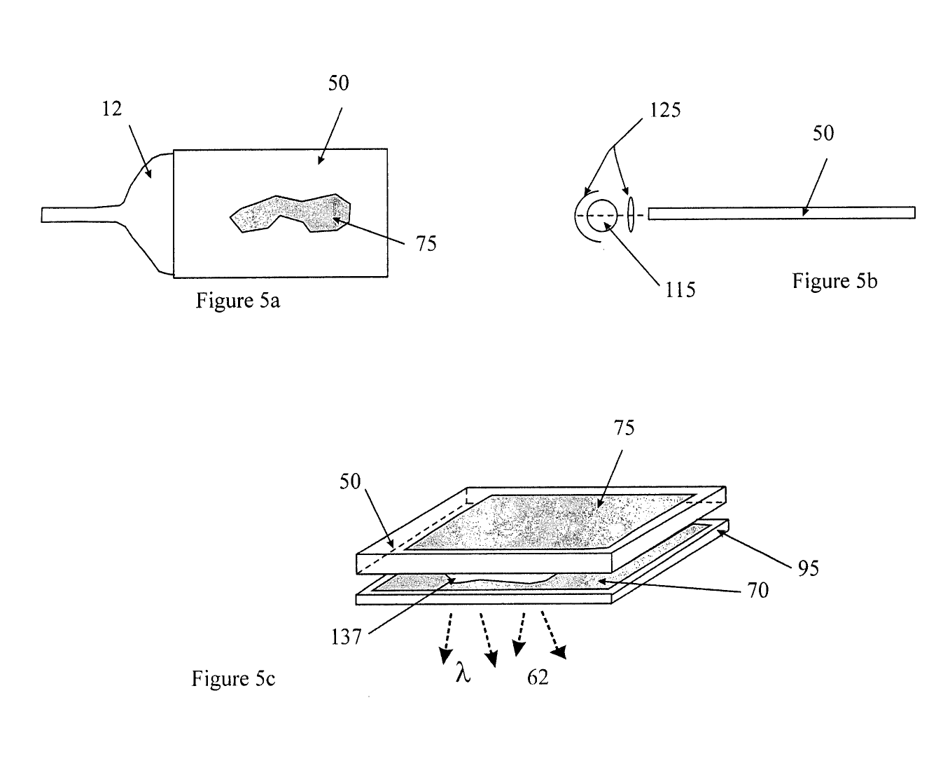

In the prior discussions, it was assumed that the light therapy device of the present invention would be used by applying a light extraction layer 75 to a surface of the light guide substrate, such as a reflective diffuser on the outer surface 58 or a transmissive micro-structured layer on the inner surface 60, where the shape, size, and position of the light extractor can be optimized relative to the treatment area. However, an alternate construction can be used, as depicted in FIG. 5c, in which the light extraction layer 75 covers all or most of a surface of the light guide substrate 58, and in which a mask 95 is applied between the inner surface and the tissue. In this case, mask 95 nominally includes an internal patterned aperture 137 that corresponds to the treatment area, through which the therapeutic light travels. Mask 95 can be light absorptive or light reflective. As shown in FIG. 5c, mask 95 has a reflective material 70 applied to the surface facing the light guide substrate. Mask 95 is shown offset from light guide substrate 50 for illustrative purposes, but in general, these two items would be held together in close proximity.

The coupling of the input light into light guide substrate 50 can be accomplished by a variety of means. For example, in FIG. 2, a fiber-optic cable 12 is shown, attaching to light guide substrate 50 at the center of input surface 52.

In this case, while light trapping within light guide substrate 50 will tend to homogenize the light, there will likely be a “hot spot” (an area of higher intensity) near the center of the light guide substrate 50. As most wound patterned light extraction layers 75 would likely be positioned near the center of the bandage 100, it may be desirable to have an intensity hot spot in the center region of the light guide substrate 50. On the other hand, the light guide substrate 50 can be more uniformly filled with light if need be. For example, in the top view of FIG. 5a, fiber-optic cable 12 is a “circle to line converter”, with the ensemble of individual optical fibers at the output end of fiber-optic cable 12 distributed over a long linear region. As another example, depicted in the cross sectional view of FIG. 5b, light source 115 is a linear light source (extending into the paper), complemented by beam shaping optics 125, which couple the output light into the light guide substrate 50. Light source 115, for example, might be a cold cathode fluorescent lamp (CCFL), a neon type tube lamp, or an elongated tungsten halogen filament lamp. Beam shaping optics 125 could include a reflector and a lens, as well as optical filtering (not shown).

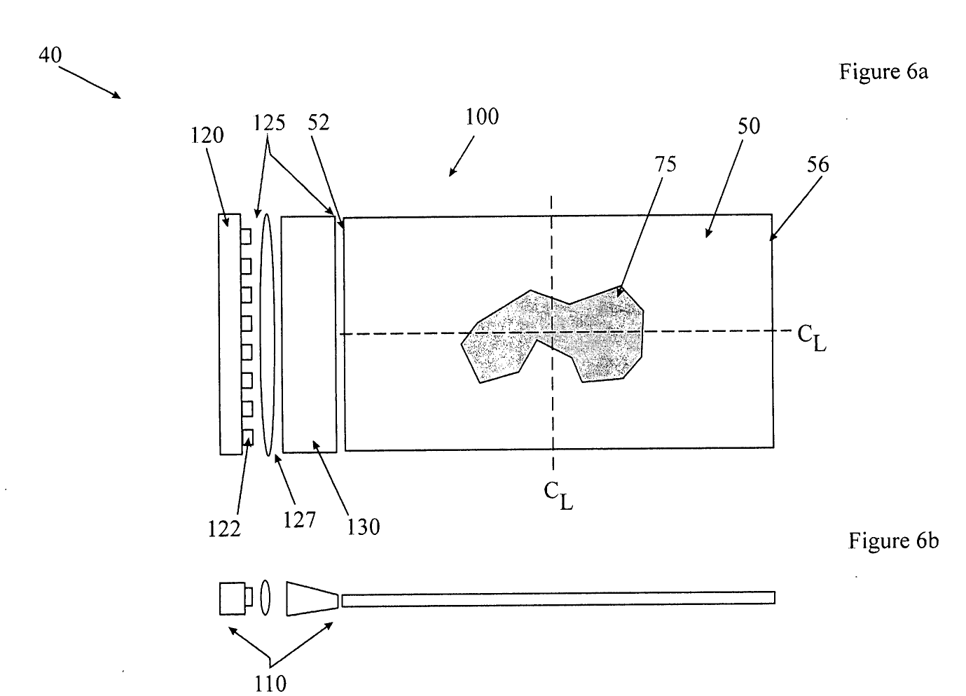

Another embodiment of light guide therapy pad (or bandage) 100 is depicted in FIGS. 6a-6b. Light therapy device 40 nominally comprises a light source module 110 and light guide substrate 50, whereas bandage 100 includes light guide substrate 50 and perhaps a portion of the light source module 110, depending on how design modularity is accomplished. Light source module 110 nominally comprises light source array 120 and beam shaping optics 125. Mounting means (not shown) would be provided to hold these various components in their proper relationships with respect to each other.

For this device, a light source array 120 is shown, which could comprise 1 to N individual light emitters 122. For example, if a fiber-optic cable 12 were branched into N smaller fiber bundles, then light emitters 122 would represent the output end of these fiber-optic bundles. More likely, the plurality of light emitters 122 represent a series of laser diodes, or light emitting diodes (LEDs), or combinations thereof. As such, the lasers or LEDs could be discretely packaged semiconductor type devices. Molded-in LEDs on flex circuits could also be used, as is done in the display industry (for example, by Global Lighting Technologies Inc.). Discrete laser diodes and LEDs are available from numerous companies, including Spectra-Physics, Coherent, SuperLum Diodes Ltd. Lumileds, Cree, Osram, or Nichia. The ensemble of laser emitters 122 could also represent a monolithic array, such as a laser diode array (although such an array would not likely extend the full width of the light guide substrate 50). The plurality of light emitters 122 could also be provided by other light source technologies, such as organic LEDs (OLEDs), polymer LEDs (P-LEDs), or thin film electroluminescent (TFEL) emitters.

Considering again FIGS. 6a-6b, the light emitted from light emitters 122 can be coupled into light guide substrate 50 by beam shaping optics 125, which are shown as comprising lens 127 and optical coupler 130. Lens 127 may represent a single lens, a lens system, or other optical elements with optical power. For example, lens 127 could represent a field lens, such as Fresnel lens. Lens 127 would have a focal length determined by the preferential light distribution sought within light guide 50. For example, lens 127 could have a focal length that would cause light to be focused towards the intersection of the two light guide center lines (CL), or a focal length that would correspond to the far end (end surface 56) of the light guide substrate 50. Lens 127 could also include optical power, for example from a cylinder lens, which would help to couple light into the near end (input surface 52) of light guide substrate 50. Beam shaping optics 125 could also include an optical coupler 130, such as a non-imaging optical light concentrator such as a tapered bar or a compound parabolic concentrator (CPC) for enhancing the efficiency of coupling input light into the light guide substrate 50. Beam shaping optics 125 could also comprise a lenslet array, where there may be a lenslet for a light emitter 122 or for a group of light emitters 122. It should be understood that beam shaping optics could also further comprise other optical elements such as spectral filters and optical polarizers.

The light guide therapy device 40 depicted in FIG. 6a-6b represents an alternate embodiment to the device shown in FIG. 2 in other ways than just the configuration of the light source and the means for optical coupling into the substrate 50. Considering again the light therapy device 40 of FIG. 2, the drive unit 14 (which includes the light source) is separate and distinct from the light guide bandage 100, with the two joined by fiber-optic cable 12. In order to facilitate ease of use by the clinician and the patient, it would be optimal if the light guide bandage 100 could be disconnected from the drive unit 14. This modularity could be provided numerous ways, but optimally, the fiber-optic cable 12 has an interface connector (not shown). Preferably this connector would be located at the juncture of the fiber-optic cable 12 and the light guide substrate 50, or alternately at some short distance (˜25 mm, for example) prior to the juncture of the fiber-optic cable 12 and the light guide substrate 50. In that latter case, light guide bandage 100 would be assembled with a short length of fiber-optic cable 12 protruding from it, which would then mate to the longer cable extending from the drive unit 14.

In this context, the light guide therapy device 40 of FIGS. 6a-b can represent another approach to modularity. Certainly this light therapy device 40 can include a separate drive unit 14, much as depicted in FIG. 2. However, as the light therapy device 40 in FIGS. 6a-6b is designed to be compact, and preferably utilize small light sources such as LEDs, then the drive unit could be incorporated somewhere within light source module 100. For example, the functions of controller 20 could be provided by a small circuit board or a flexible thin film circuit could be included in the assembly for light source array 120. Presumably battery power would also be included. If this assembly is sufficiently small and light, it could an integral permanent assembly with light guide substrate 50. However, as the goal is to provide a low cost light therapy bandage which is easy for the patient to wear for prolonged periods of time, and which further may have the portion that is in contact with the treatment area replaceable, if not disposable, then the various further concepts for modularity maybe utilized. For example, the light therapy bandage 100 could comprise light guide substrate 50 and the various coatings and layers (per FIGS. 3a-b) and little else. In that case, modularity could be enabled by having the bandage separate from the light source module 110 at the juncture of the light coupling means 130 and the light guide substrate 50. This has the advantage that light guide substrate 50 is nominally a sheet material which can be readily fabricated. On the other hand, changing the light guide substrate 50 could require a clinician to align the flexible sheet light guide substrate 50 to the light source module 110. Attaining that alignment over the width of the substrate 50, while providing efficient light coupling, could be difficult, particularly in the field.

Alternately, modularity could be provided by having light therapy bandage 100 include both substrate 50 and light coupling means 130. Light coupling means 130 could be fabricated separately, and then adhered or fused to the light guide substrate 50. Alternately, bandage 100 could be partially made using an extrusion process, where light coupling means 130 is formed at the end of substrate 50 as one contiguous piece. Light coupling means 130 would still be part of the beam shaping optics 125, but not part of the light source module 110, as it was originally defined. In this case, the alignment of the bandage 100 to the incident light from the light source module would be significantly easier, because the incoming light beam would still be relatively large, as would the light coupling means 130. This would also likely simplify the design of the mechanical mounting interface structures provided for light source module 110 and bandage 100, as well as improving the robustness of the mechanical design. Certainly other design variations can be considered as means to provide modularity for the light therapy device 40 of the present invention.

It is noted that light guide substrate 50 is shown in the figures as having a constant nominal thickness T over the length and width of the sheet. Alternately, substrate 50 can have a wedged profile, with the thicker end corresponding to input surface 52. If the input end is sufficiently thicker, the potential need for a light coupling means 130 may be obviated. Alternately, providing a wedge in the sheet material towards the input surface 52 may ease the mechanical interface to the optical coupling means 130.

Certainly the light guide therapy device 40 would be designed to be adaptable to facilitate treatment of a variety of conditions. For example, the bandage 100 could have a square form factor, as small as 2.5 in×2.5 in., or as large as 10 in.×10 in., or a rectangular form factor, such as 8 in.×20 in. The thickness T of substrate 50 would nominally be ˜1-2 mm, although it could be as little as 0.1-0.5 mm, as long as the required flexibility is achieved. It may also be desirable that the light guide substrate 50 be fabricated from a material that is extensible, so that the bandage can be stretched and wrapped (for example around a limb). The clinician may also encounter circumstances where it would be desirable to modify the outward shape of the light guide substrate, as wrapping the bandage, even if flexible and conformal, around some portion of a patient's body, may not be an adequate solution. With respect to this issue, FIG. 4d depicts a light guide substrate that has been modified with cut edges 65. Unfortunately, cutting the edges in this manner will likely cause light to leak out the new edge surfaces. Potentially the clinician could stop this light leak by applying a reflective layer, such as the previously mentioned 3M ESR film, to the edges. This could be an awkward activity for the clinician to undertake, particularly in the field. Alternately, an easy to apply reflective material 70, such as a quick curing metal (silver, for example) impregnated epoxy or adhesive could be applied along the edges. Although light guide bandage 100 has been generally depicted in the various figures as comprising a substrate 50 with sharp corners, it should be understood that the devices could be initially fabricated with rounded corners, which might aid comfortable application onto a patient.

The operational wavelength could be variable, depending on the condition being treated. For example, the bandages could emit blue light for treating jaundice in infants. Alternately, the bandages could be designed to emit red light (such as 632 nm or 670 nm) or infra-red light (such as 840 nm), or the combination thereof. Numerous academic studies have demonstrated enhanced healing effects for conditions such as burns, pressure ulcers, and chronic pain, with application of red and/or IR light. This is partially because most human tissue has a transmission window, from ˜630 nm to ˜1200 nm where light can penetrate ˜3-4 mm into the tissue. Furthermore, various bio-chemicals, such as cytochrome oxidase, have been shown to be particularly photo-reactive to incident light in that spectral range. The energy received by such photo-chemicals can then be used in various ways to enhance healing. Also, as some studies have suggested that laser light may provide more efficacious healing than does incoherent light (such as that from LEDs), it is emphasized that the light source can comprise one or more lasers (such as laser diodes), which can be used by themselves or in combination with incoherent light sources. It is also noted, that in many of the published light therapy studies, the applied optical intensity ranges between 5-100 mw/cmˆ2. The light guide bandage of the present invention is generally intended to meet these apparent optical power needs. However, given that the light guide bandage is intended to stay on a patient for a prolonged duration, then it is intended to employ longer exposure times at lower power levels. The lower power levels do need to fall within a range where reciprocity applies, and lower power levels still provide a beneficial effect, rather than little or no effect. For example, biological time constants or threshold effects may limit the lower level of light exposure.

Controller 20 nominally provides intensity control, as well as light modulation (nominally at frequencies in the 5 Hz-5 kHz range) and repeat treatment programming capability. Controller 20 could also include intensity calibration functionality, as well as data management for any feedback or bio-sensing capabilities that might be built into the bandage. Depending on the circumstances, it may or may not be desirable to allow the consumer or patient to control the operation of the light therapy bandage of the present invention. It should be understood that the light therapy device of the present invention could be used not only for light therapy, but also for photodynamic therapy (PDT).

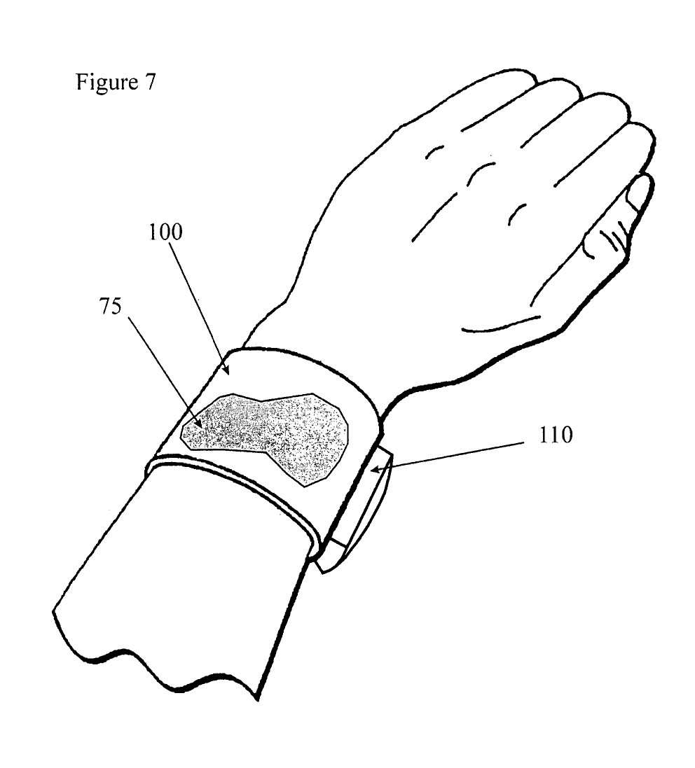

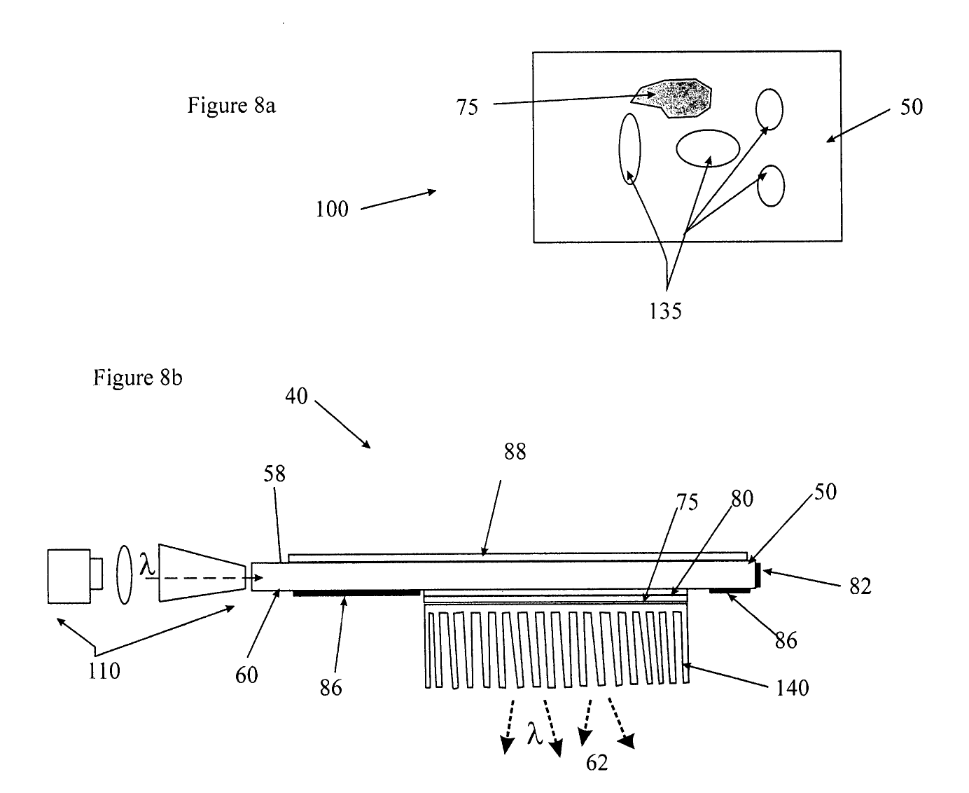

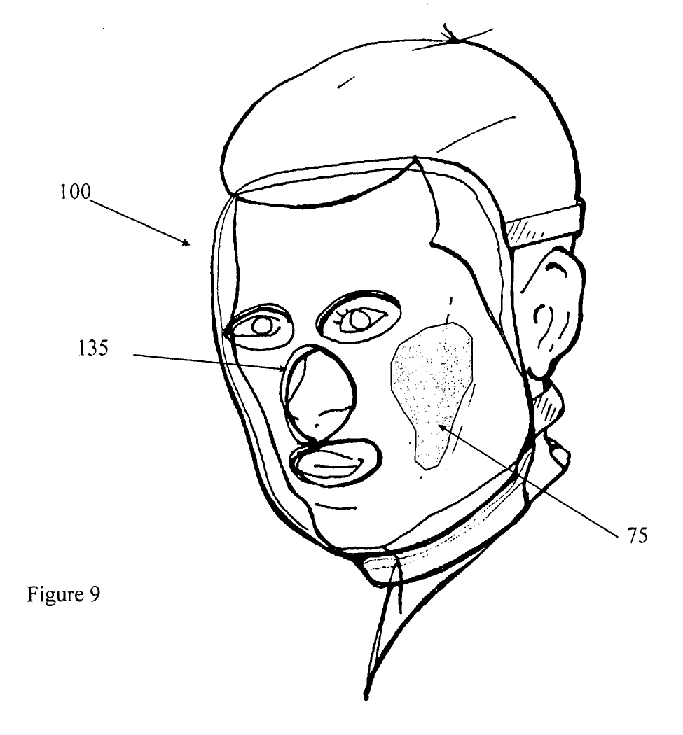

To illustrate the general concept of the light therapy device 40 of the present invention, FIG. 7 is provided, in which a light guide bandage 100 is shown wrapped around a patients arm. Light guide bandage 100 may also be equipped with apertures 135 (see FIG. 8a) to allow the passage of air flow and perspiration, or to allow clearance for appendages (fingers, for example). The inside edges of apertures 135 could be coated with a reflective layer, such as the metallized epoxy that was mentioned previously. FIG. 9 then shows a general concept of the light guide bandage 100 as a facial mask attached to a patients head with straps. In this instance, the apertures 135 more readily allow the patient to breathe, talk, see, and smell while wearing the mask. For example, the clinician could use a digital camera to capture an image of the patients face, then down load the image onto a light guide template, which would determine how the apertures 135 should be shaped and positioned. In this case, light guide bandage 100 may not only be flexible and conformal, but may also be molded to better follow the complex features of the tissue it is applied to. FIG. 8b shows another alternative concept where the light guide therapy device 40 is equipped with a light guide adaptor 140 that protrudes from the inner surface 60 of the substrate 50. For example, a light guide adaptor 140 might facilitate treatment in circumstances where there is dense hair, such as a cranial treatment or a veterinary treatment (where there is fur), in which a goal is to avoid shaving the hair off while still allowing treatment.

The conceptual designs for the light therapy device of the present invention have been discussed with emphasis on providing a low cost, customizable, light therapy bandage that is modular. In part, the modularity is emphasized as an enabling means to allow the clinician to adapt the bandage to the changing characteristics of the wound over time. In part also, the emphasis on modularity and low cost is also to enable the clinician to readily change and adapt the bandage in the field. In this latter context, it needs to be understood that a clinician may change normal bandages on a wound 2-3 times per week. Thus the light therapy bandage needs to have sufficient ease of use, as well as well as a low enough cost, that its use is economically feasible. If the cost of the entire device was sufficiently low, then the entire bandage, light source included, could be discarded after use. But as the light source module 110 or the drive unit 14 will likely have some significant costs associated with them, it is likely desirable to have a modular design, where the light guide bandage can be separated from the light source. Alternately, the light source emitters, can be formed directly onto the substrate 50. For example, either patterned organic LEDS (O-LEDs) or polymer LEDs (P-LEDs) could be fabricated at an edge or side of substrate 50, so that the emitted light is directly coupled into the substrate. Perhaps, the molded-in LEDs on flex circuits, integrated directly on an edge of the substrate 50, could also be used. In such instances, the modularity of light therapy bandage 100 could be compromised in order to have a fully integrated light source and light guide, provided that the cost of the combination unit was still sufficiently low, that the bandage 100 could be cost effective. The advantage in this case, is that with the light source module 100 effectively integrated into the bandage with a very low profile, the thickness and rigidity of the bandage 100 at the light input might be minimized, potentially making the device usage easier for both the patient and the clinician.

The light therapy bandage 100 of the present invention is generally conceived to have a combination of adaptability, physical flexibility, modularity, and low cost, that a clinician would readily apply it to a patient for an extended period of time (for example, several days), during which the device would likely operate according to some predetermined protocol. For the light therapy bandage of the present invention to have the greatest utility, it should be integrated with other bandage elements. Preferably, bandage 100 could be combined with other types of bandages or dressings, such as hydro-colloidals, alginates, or anti-biotic silver bandages. In such a case, these other bandages or dressings would provide required functions to keep the wound moist and suppress infections, and bandage 100 could slip into a sleeve or pocket in one of the other bandages. Attachment feature 86, which could be an adhesive or Velcro, could be used to assist such combinations. In such instances, it would likely be required that any bandages or dressings that are intervening between bandage 100 and the wounded tissue be sufficiently transparent at the treatment wavelengths, that the treatment light can effectively reach the tissue. Of course, exudates (fluid, cells or other substances that have been slowly exuded, or discharged, from cells or blood vessels) may be present and reduce the effectiveness of the light therapy from the bandage 100. Thus, it is generally preferable that bandage 100 can be cleaned. Therefore, bandage 100 may be combined with other types of dressings, such as vacuum sponges, that help remove exudates. Additionally, bandage 100 may be equipped directly with the previously mentioned tissue interface layers 84 (preferably transparent) that provide the needed features of modern bandages or dressings, such as alginate or anti-bacterial silver functionality. In that case, bandage 100 may include a foam or gel that contacts the tissue. Preferentially, bandage 100 can be cleaned and re-used on the patient. Light guide bandage 100 likely also needs to be waterproof and crushable, as well as non-allergenic. Some portion of the bandage or dressing including bandage 100 needs to be moisture permeable and breathable. The light source and associated drive electronics could be re-used, perhaps rented or leased, or sold to the clinician or consumer for ongoing use.

It is noted that light guide therapy pad 100 is also generally similar to the light guides used in backlights for laptop computer and mobile phone displays. Display backlighting systems typically comprise a light source, a light guide member, and a light extraction means. For example, the light source is typically a cold cathode fluorescent lamp or an array of LEDs that are coupled into one end of the thin sheet light guide. The light guide substrate can likewise be equipped with a light extraction layer, such as a light diffusion layer, or a prismatic sheet. In many cases, the light extraction means comprises a volume diffusion mechanism, such as beads or bubbles that act as light scatterers, and which are imbedded in the light guide itself. There are numerous prior art patents known in the art for display backlights, including U.S. Pat. No. 5,005,108 (Pristah et al.); U.S. Pat. No. 6,079,838 (Parker et al.) and U.S. Pat. No. 6,712,481 (Parker et al.). The most important difference for the present invention, and in particular light guide therapy pad 100, as compared to the prior art known from display backlighting, is that the display backlights designs are motivated to provide uniform spatial and uniform angular light output over nearly the full length and width of the light guide panel. In particular, in the backlight applications, the goal is to reduce the spatial non-uniformity over the display to a few percent, so that the user is nominally unaware of any residual variation within the viewing area. Many backlight display designs employ spatially variant or patterned diffusers, micro-structures, or deformities, but with the goal to transform a non-uniform light input (often at one end) into a spatially uniform light output over nearly the full area of the light guide. Likewise, the goals in backlight design often include control the horizontal and vertical angular directionality of the output light, to maximize light efficiency within the likely viewing angles (for example +/−15° vertically and +/−30° horizontally) to allow the user to view the screen with minimal change over some angular range, as for example, the user turns his or her head. The display backlights, which are usually illuminating a liquid crystal panel, are also usually equipped with other layers, such as color filters (and particularly color filter arrays) and contrast enhancement layers, so that the display provides high contrast full color illumination. As the light guide therapy device of the present invention does not employ addressed modulated high-resolution pixels, neither color registered color filter arrays nor contrast enhancement layers are needed.

The light guide therapy pad 100 of the present invention is different and distinct from the display backlights in several regards. In particular, the light extraction means (light extraction layer 75 or mask 95) of the present invention is not nominally applied to the entire surface area of the light guide, but is only applied to a smaller portion corresponding to one or more treatment areas. Therefore, spatial uniformity of the light distribution exiting the light guide 100 is not a priority, and may not even be desirable (per FIGS. 4a, 4b). Likewise, the light guide 100 of the present invention is not designed with an emphasis on controlling the angular spread of the exiting light. In general, the design goal would be to have the therapeutic light 62 emerge over some range of angles, from normal existence out to θmax, where θmax is likely between 20-45°. However, separate control of angular emissions in the two meridians (“horizontal” and “vertical”) is not required. Importantly also, the light guide therapy pad of the present invention is designed to facilitate customization of the light extraction to match the treatment area, as well as to facilitate the potential disposability or reusability of all or a portion of the light guide therapy pad 100. In this context, the pad 100 is designed so that the clinician can apply a light extraction layer 75 or mask 95 to the light guide substrate 50, as well as remove and replace the light guide bandage 100 relative to the light source module 110 or the drive unit 14. In contrast, display backlights are designed as factory integrated packages, with the light source (such as the CCFL) and the light guide held in a fixed relationship, without any intent for user modification. The user is not expected to change the light guide relative to the light source, or to alter the light extraction capabilities of the light guide. Additionally, while the light guide therapy pad 100 of the present invention may be equipped with secondary layers and functions, such as anti-biotic layers, adhesive layers (to the tissue), non-stick layers, or bio-sensing layers, these layers are functionally different than the color filter and contrast enhancement layers provided in display backlights.

Throughout the previous discussions in which the present invention has been described, the focus has been directed towards the treatment of wounds, such as chronic wounds, as exemplified by pressure ulcers. Certainly the device of the present invention can be used to treat other types of chronic wounds (such as diabetic ulcers or venous stasis ulcers), as well as acute wounds (such as cuts and incisions), burns, jaundice, and various skin conditions (acne, psoriasis, fine lines and wrinkles, etc.), as well as other conditions not listed here. Under the appropriate circumstances, the device of the present invention might even be used for internal (such as body cavity) treatment applications.

The invention has been described in detail with particular reference to a presently preferred embodiment, but it will be understood that variations and modifications can be effected within the scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

### PARTS LIST

10 fiber-optic pad
12 fiber-optic cable
14 drive unit
16 connector
20 controller
22 display
24 front panel
40 light therapy device
50 light guide substrate
52 input surface
54 side surface
56 end surface
58 outer surface
60 inner surface
62 therapeutic light
65 cut edges
70 reflective material
75 light extraction layer
80 optical coupling layer
81 other optical layers
82 mirror layer
84 tissue interface layer
86 adhesive layer
88 cover
95 mask
100 light guide therapy pad
110 light source module
115 light source
120 light source array
122 light emitters
125 beam shaping optics
127 lens
130 optical coupler
135 apertures
137 patterned aperture
140 light guide adaptor

1. A light therapy device for delivering light energy to a portion of a patient's body, comprising:
a) a light source, comprising one or more light emitters for providing input light;
b) a light coupling means for directing said input light into a light guide;
c) a flexible optically transparent light guide material, which comprises said light guide;
d) a light extraction means which is applied to a surface of said light guide material; wherein said light extraction means is positioned to provide light therapy treatment to one or more localized areas of said patient's body; and
e) a control means, which controls a light dosage relative to intensity, wavelength, modulation frequency, repetition, and timing of treatments.

2. A light therapy device as in claim 1 wherein said light extraction means is a reflective optical diffuser layer.

3. A light therapy device as in claim 1 wherein said light extraction means comprises a micro-structured transmissive optical layer.

4. A light therapy device as in claim 1 wherein said light extraction means includes a light extraction layer and a mask.

5. A light therapy device as in claim 1 wherein said light extraction means is customizable to generally conform to the size and shape of the areas to be treated.

6. A light therapy device as in claim 1 wherein a clinician examines the wounds and transfers the shape and size information to said light extraction layer, and modifies said light extraction layer to aid illumination of the wound area, and then attaches the customized light extraction layer to said light guide.

7. A light therapy device as in claim 6 wherein a digital camera is used to customize said light extraction means.

8. A light therapy device as in claim 1 wherein said light guide has apertures to conform said light guide to body structures.

9. A light therapy device as in claim 1 wherein edges or corners of said light guide are modified to conform to local features of said patients' body.

10. A light therapy device as in claim 9 wherein a reflective epoxy or adhesive, such as a silver impregnated epoxy, is applied to said edges.

11. A light therapy device as in claim 1 wherein the device comprises polarization layers to irradiate said localized areas with polarized light.

12. A light therapy device as in claim 1 wherein said light source comprises one or more LEDs, lasers, lamps.

13. A light therapy device as in claim 1 wherein said light source emits light within a spectral range of 400-1500 nm.

14. A light therapy device as in claim 1 wherein said light source emits red or IR light.

15. A light therapy device as in claim 1 wherein said light guide material comprises a non-woven polymer material, such as a polyurethane, elastomer, or acetate.

16. A light therapy device as in claim 1 wherein said light source is temporally modulated within a frequency range encompassing 5 Hz to 5 kHz.

17. A light therapy device as in claim 1 wherein said light coupling means comprises beam shaping optics comprising a tapered light guide, a Fresnel lens, a cylinder lens, a lenslet array, or other appropriate optical components.

18. A light therapy device as in claim 1 wherein said light guide can be removed from said light source and replaced by another light guide.

19. A light therapy device as in claim 1 wherein said light extraction means includes an adaptor for hair.

20. A light therapy device as in claim 1 wherein said light source includes spectral filters.

21. A light therapy device as in claim 1 wherein said light guide is coated with hygienic layers, antibiotic layers, bio-chemical marker, or anti-tacky outermost surface.

22. A light therapy device as in claim 1 wherein light guide is a sheet, which can be of uniform thickness or wedged, with the thick end towards said light source.

23. A light therapy device as in claim 1 wherein said light guide has a pre-formed or pre-molded shape.

24. A light therapy device as in claim 1 wherein said light guide is shaped like a face.

25. A light therapy device as in claim 1 wherein said light therapy device is modular, and can be disassembled between said light source and said light guide.

26. A light therapy device as in claim 1 wherein said light therapy device provides bio-sensors.

27. A light therapy device for delivering light energy to a portion of a patient's body comprising:
a) a light source, comprising one or more light emitters for providing input light;
b) a coupler for directing said input light into a light guide;
c) a flexible, optically transparent, light guide material, which comprises said light guide;
d) a light extraction layer which is applied to a surface of said light guide material, such that light exits said light guide; wherein said light extraction layer is customized for shape, size, and position to provide light therapy treatment to one or more localized areas of said patient's body;
e) a controller, which controls the light dosage relative to intensity, wavelength, modulation frequency, repetition and timing of treatments; and
wherein said light therapy device is modular, and can be disassembled between the light source and the light guide.

28. A light therapy bandage device for delivering light energy to a portion of a patient's body, comprising:
a) a light source, comprises an array of light emitters for providing input light;
b) a light coupler for directing said input light into a light guide;
c) a flexible optically transparent light guide material, which comprises said light guide;
d) a light extraction layer which is applied to a surface of said light guide material, such that light exits said light guide and is available for a light therapy; wherein said light extraction layer is customized for shape, size, and position to provide light therapy treatment to one or more localized areas of said patient's body;
e) a controller, which controls the light dosage relative to intensity, wavelength, modulation frequency, repetition and timing of treatments, and other parameters; and
wherein said light therapy device is modular, and can be disassembled between the light source and the light guide.

29. A light therapy bandage device for delivering light energy to a portion of a patient's body, comprising:
a) a light source, comprises an array of light emitters for providing input light;
b) a light coupler for directing said input light into a light guide;
c) a flexible optically transparent light guide material, which comprises said light guide;
d) a light extraction layer which is applied to a surface of said light guide material, such that light exits said light guide and is available for a light therapy;
e) a controller, which controls the light dosage relative to intensity, wavelength, modulation frequency, repetition and timing of treatments, and other parameters;
wherein said light extraction layer is pre-assembled onto said light guide; and
wherein said light therapy bandage is applied to said patient's body such that said light extraction layer is positioned to provide light therapy treatment to one or more localized areas of said patient's body.

30. A light therapy bandage device for delivering light energy to a portion of a patient's body, comprising:
a) a light source, comprises one or more light emitters for providing input light;
b) a light coupler for directing said input light into a light guide;
c) a flexible optically transparent light guide material, which comprises said light guide;
d) a light extraction layer which is a reflective optical diffuser applied to the outer surface of said light guide material, such that light exits said light guide and is available for a light therapy; wherein said light extraction layer is customized for shape, size, and position to provide light therapy treatment to one or more localized areas of said patient's body; and
e) a control means, which controls the light dosage relative to intensity, wavelength, modulation frequency, repetition and timing of treatments, and other parameters.

31. A light therapy bandage device for delivering light energy to a portion of a patient's body, comprising:
a) a light source, comprises one or more light emitters for providing input light;
b) a light coupler for directing said input light into a light guide;
c) a flexible optically transparent light guide material, which comprises said light guide;
d) a light extractor which is a transparent micro-structured optical layer applied to a surface of said light guide material, such that light exits said light guide and is available for a light therapy; wherein said light extractor is customized for shape, size, and position to provide light therapy treatment to one or more localized areas of said patient's body; and
e) a controller, which controls the light dosage relative to intensity, wavelength, modulation frequency, repetition and timing of treatments, and other parameters.

32. A light therapy bandage device for delivering light energy to a portion of a patient's body, comprising:
a) a light source, comprises one or more light emitters for providing input light;
b) a light coupler for directing said input light into a light guide;
c) a flexible optically transparent light guide material, which comprises said light guide;
d) a light extractor which comprises a micro-structured optical layer applied to a surface of said light guide material, and a mask, such that light exits said light guide and is available for a light therapy; wherein said mask portion of said light extractor is customized for shape, size, and position to provide light therapy treatment to one or more localized areas of said patient's body; and
e) a controller, which controls the light dosage relative to intensity, wavelength, modulation frequency, repetition and timing of treatments, and other parameters.

33. A method for providing light therapy using a light guide bandage for treatment of one or more areas of a patient, comprising:
a) examining the treatment areas to determine the condition of the tissues therein;
b) determining the size and extent of the treatment areas to be treated with light therapy;
c) customizing a light extraction layer onto a light guide bandage in accordance with the treatment areas;
d) combining said light guide bandage with a light source and a controller;
e) assembling the light guide bandage with other bandage elements; and
f) setting control parameters for the light therapy.

34. A method for providing light therapy using a light guide bandage for treatment of one or more areas of a patient, comprising:
a) examining the treatment areas to determine the condition of patient tissues;
b) determining the size and shape of the treatment areas to be treated with light therapy;
c) providing a light guide bandage, which includes a light source and a controller, with a light extraction layer that generally corresponds to said treatment areas;
d) assembling said light guide bandage with other bandage elements; and
e) setting control parameters for the light therapy.

35. A method for providing light therapy as in claim 34, wherein said light guide therapy device delivers repeated controlled treatments over a course of time.

36. A method for providing light therapy as in claim 34, wherein at least part of said light guide bandage is modified or replaced during treatment of said patient.

37. A light therapy device for delivering light energy to a portion of a patient's body, comprising a light source that consisting of one or more light emitters for providing input light to a light guide, wherein said light guide comprises a flexible optically transparent light guide material, wherein said light guide material is provided with a light extraction means that is spatially patterned across one or more portions of said light guide so as to direct light out of said light guide to then provide light therapy treatment to one or more localized areas of said patient's body.

38. A light therapy device for delivering light energy to tissues requiring treatment, comprising a light source consisting of one or more light emitters for providing input light to a flexible transparent optical substrate material, wherein said light therapy device is provided with a light extraction means that is spatially patterned across one or more portions of said device so as to direct light out of said device to then provide light therapy treatment to one or more localized areas of said tissues.

39. A light therapy device for delivering light energy to a portion of a patient's body, comprising:
a) a light source, comprising one or more light emitters for providing input light for a light guide;
b) a flexible optically transparent light guide material, which comprises said light guide; and
c) a controller means, which controls a light dosage emitted from said light therapy device;
wherein said light guide material is provided with a light extraction means that is spatially patterned across one or more portions of said light guide so as to direct light out of said light guide to provide light therapy treatment to one or more localized areas of said patient's body.

40. A light therapy device for delivering light energy to tissues requiring treatment, comprising:
a) a light source, comprising one or more light emitters for providing input light for a light guide;
b) a flexible optically transparent light guide material, which comprises said light guide;
c) a tissue interface layer, which is attached to a surface of said light guide, and which is proximate to tissues; and
d) a controller means, which controls a light dosage emitted from said light therapy device;
wherein said light guide material is provided with a light extraction means that is spatially patterned across one or more portions of said light guide so as to direct light out of said light guide to provide light therapy treatment to one or more localized areas of said tissues.

41. A light therapy device for delivering light energy to a portion of a patient's body, comprising:
a) a light source, comprising one or more light emitters;
b) a light coupling means comprising one or more optical fibers for coupling said input light from said light source into a light guide;
c) a flexible optically transparent light guide material, which comprises said light guide; and
d) a controller means, which controls a light dosage emitted from said light therapy device;
wherein said light guide material is provided with a light extraction means that is spatially patterned across one or more portions of said light guide so as to direct light out of said light guide to provide light therapy treatment to one or more localized areas of said patient's body.

42. A light therapy device as in claim 1 wherein said light coupling means comprises one or more optical fibers for coupling said input light from said light source into said light guide.
